(19) United States
(12) Patent Application Publication
(10) Pub. No.: US 2024/0082440 A1
(43) Pub. Date: Mar. 14, 2024

(54) SYSTEM AND METHOD FOR DISRUPTION OF MACROMOLECULES USING MID- AND FAR- INFRARED

(71) Applicant: SIGNIFY HOLDING B.V., EINDHOVEN (NL)

(72) Inventors: MARC ANDRE DE SAMBER, LOMMEL (BE); KARS-MICHIEL HUBERT LENSSEN, VELDHOVEN (NL); PETER DEIXLER, (US); WILLEM DIRK VAN DRIEL, 's-Hertogenbosch (NL); RÉMY CYRILLE BROERSMA, EINDHOVEN (NL)

(21) Appl. No.: 18/273,847

(22) PCT Filed: Jan. 13, 2022
(86) PCT No.: PCT/EP2022/050638

§ 371 Date: Jul. 24, 2023

Related U.S. Application Data
(60) Provisional application No. 63/141,593, filed on Jan. 26, 2021.

(30) Foreign Application Priority Data
Feb. 25, 2021 (EP) 21159165.6

(51) Int. Cl. A61L 2/08

(52) U.S. Cl. CPC A61L 2/085; A61L 2202/11; A61L 2202/14; A61L 2202/16; A61L 2209/111; A61L 2209/12

(57) ABSTRACT

A method (100) for disinfection using a lighting system (200), comprising: •determining (140) a mid-infrared (IR) and/or far-IR wavelength configured to disrupt a target macromolecule of a target pathogen, wherein the target macromolecule is DNA, RNA, and/or •a protein of the target pathogen; and exposing (150), via a light source (210) of the lighting system (200), •exposing (150), via a light source of the lighting system (200), the target pathogen to the determined mid-IR and/or far-IR wavelength, wherein the target macromolecule is directly disrupted and the target pathogen is neutralized by the exposure; and •detecting (160), by a sensor (224) of the lighting system (200), neutralisation of the target pathogen by exposure.

### FIELD OF THE INVENTION

The present disclosure is directed generally to systems and methods for disinfection using a lighting system.

### BACKGROUND

Recent pathogen outbreaks have shown that there is a continued demand for more powerful, more efficient, faster, cheaper, and more readily available methods to fight infectious diseases. For example, pathogens such as viruses can be transmitted via short distance particle transmission between humans, e.g. during coughing or sneezing, but also significantly via contaminated surfaces. Many viruses can easily survive for days on surfaces such as tables, door handles, paper, and other commonly utilized surfaces.

Since these viruses are transmitted between humans and by surface contact, some of the riskiest places for viral infection are places where large numbers of already infected people intersect with caregivers as well as other patients with lowered immunological status, such as in operation rooms theatres, medical examining rooms, common areas in elderly homes, and other locations.

Current methods to disinfect these locations and the surfaces therein are mainly based on cleaning with water and soap and/or with chemicals such as alcohol and the like. A more recent disinfection method is the introduction of non-touch UV-C based light disinfection systems, mainly based on conventional UV-C light tubes, Excimer lamps, or

Xenon lamps.

However, these legacy disinfection methods have major drawbacks. Manual contact cleaning with chemicals might lead to surfaces that are forgotten or insufficiently well cleaned. Often the chemicals used in these cleanings are harmful to the environment and to the people working with them. Manual cleaning procedures can vary considerably among hospitals, and it has been suggested that less than 50% of patient room surfaces are properly cleaned.

Further, exposure to UV-C disinfection light above a threshold dose limit is very harmful for humans, including potentially resulting in injury to eyes and/or skin.

### SUMMARY OF THE INVENTION

Accordingly, there is a continued need in the art for efficient and effective disinfection of locations and surfaces using environmentally-friendly systems.

The present disclosure is directed to inventive methods and systems for disinfection using a lighting system. Various embodiments and implementations herein are directed to a system comprising a light source that is capable of emitting light in at least the mid-infrared (IR) and/or far-IR range. The emitted light is utilized to disinfect a target surface or volume of air by targeting a pathogen. A specific mid-IR and/or far-IR wavelength configured to disrupt a target macromolecule of a target pathogen is determined. According to an embodiment, the macromolecule is DNA, RNA, and/or a protein of the target pathogen. Once the wavelength is determined, the light source exposes the volume of air or surface to the specific mid-IR and/or far-IR wavelength such that the target macromolecule is directly disrupted and the target pathogen is neutralized by the exposure.

Generally, in one aspect, a method for disinfection using a lighting system is provided. The method includes: (i) determining a mid-infrared (IR) and/or far-IR wavelength configured to disrupt a target macromolecule of a target pathogen, wherein the target macromolecule is DNA, RNA, and/or a protein of the target pathogen; and (ii) exposing, via a light source of the lighting system, the target pathogen to the determined mid-IR and/or far-IR wavelength, wherein the target macromolecule is directly disrupted and the target pathogen is neutralized by the exposure.

According to an embodiment, the mid-infrared (IR) and/or far-IR wavelength configured to disrupt a target macromolecule of a target pathogen is determined based on a spectroscopic property of the target pathogen.

According to an embodiment, the mid-IR and/or far-IR wavelength is determined based at least in part on molecular modeling of the target macromolecule.

According to an embodiment, the method further includes the step of eliminating at least some liquid surrounding the target pathogen prior to said exposing step.

According to an embodiment, the macromolecule is a surface protein of a virus.

According to an embodiment, exposing the target pathogen to the determined mid-IR and/or far-IR wavelength results in an intermediate product state of the target macromolecule leading to deactivation of the target macromolecule, and further comprising the steps of: determining a second mid-IR and/or far-IR wavelength configured to disrupt the target macromolecule in the intermediate product state; and exposing the target pathogen to the determined second mid-IR and/or far-IR wavelength.

According to an embodiment, the method further includes the detecting, by a sensor of the lighting system, neutralization of the target pathogen by the exposure.

According to an embodiment, the method further includes reporting neutralization of the target pathogen by the exposure.

According to an embodiment, exposing the target pathogen to the determined mid-IR and/or far-IR wavelength comprises exposing a volume of air to the light source of the lighting system.

According to an embodiment, exposing the target pathogen to the determined mid-IR and/or far-IR wavelength comprises exposing a surface which is a first distance from the light source of the lighting system, wherein the first distance comprises at least a meter.

According to an aspect is a lighting system configured to neutralize a target pathogen. The system includes: (i) a light source configured to emit a predetermined mid-infrared (IR) and/or far-IR wavelength, wherein the mid-IR and/or far-IR wavelength is configured to disrupt a target macromolecule of the target pathogen, wherein the target macromolecule is DNA, RNA, and/or a protein of the target pathogen; and (ii) a controller configured to control the light source, wherein the controller is pre-programmed with the predetermined mid-IR and/or far-IR wavelength.

According to an embodiment, the predetermined mid-IR and/or far-IR wavelength results in an intermediate product state of the target macromolecule, and wherein the luminaire is configured to emit a second predetermined mid-IR and/or far-IR wavelength, the second mid-IR and/or far-IR wavelength configured to disrupt the target macromolecule in the intermediate product state, and wherein the controller is further pre-programmed with the second predetermined mid-IR and/or far-IR wavelength.

According to an embodiment, the lighting system is configured to neutralize a target pathogen located on one or more surfaces of an environment in which the lighting system is installed.

According to an embodiment, the system further includes a temperature sensor configured to measure a temperature of one or more of the one or more surfaces, and wherein the controller is further configured to control the luminaire to: (1) stop emitting the mid-IR and/or far-IR wavelength if the measured temperature exceeds a predetermined threshold; or (2) lower an intensity of the mid-IR and/or far-IR wavelength if the measured temperature exceeds the predetermined threshold.

According to another aspect is a handheld device configured to neutralize a target pathogen. The handheld device includes: (i) a light source configured to emit a predetermined mid-infrared (IR) and/or far-IR wavelength, wherein the mid-IR and/or far-IR wavelength is configured to disrupt a target macromolecule of the target pathogen, wherein the target macromolecule is DNA, RNA, and/or a protein of the target pathogen; and (ii) a controller configured to control the light source, wherein the controller is pre-programmed with the predetermined mid-IR and/or far-IR wavelength.

According to an embodiment, the predetermined mid-IR and/or far-IR wavelength is determined based at least in part on molecular modeling of the target macromolecule.

According to an embodiment, the handheld device is configured to expose the target pathogen to the determined mid-IR and/or far-IR wavelength at a distance of 10 cm or less.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for disinfection, in accordance with an embodiment.

FIG. 2 is a schematic representation disinfection lighting system, in accordance with an embodiment.

FIG. 3 is a schematic representation of an environment comprising a disinfection lighting system, in accordance with an embodiment.

FIG. 4 is a schematic representation of an environment comprising a disinfection lighting system, in accordance with an embodiment.

FIG. 5 is a schematic representation of an environment comprising a disinfection lighting system, in accordance with an embodiment.

### DETAILED DESCRIPTION OF EMBODIMENTS

The present disclosure describes various embodiments of a lighting system configured to emit light in at least the mid-infrared (IR) and/or far-IR range. More generally, Applicant has recognized that it would be beneficial to provide a lighting system configured to target pathogens in the air and/or on one or more surfaces. A particular goal of utilization of certain embodiments of the present disclosure is to use a lighting system emitting light in the mid-IR and/or far-IR range to disrupt one or more macromolecules of a target pathogen.

In view of the foregoing, various embodiments and implementations are directed to a lighting system with one or more light sources configured to emit light in the mid-IR and/or far-IR range. A specific mid-IR and/or far-IR wavelength configured to disrupt a target macromolecule of a target pathogen is determined. According to an embodiment, the macromolecule is DNA, RNA, and/or a protein of the target pathogen. Once the wavelength is determined, the light source exposes the volume or air or surface to the specific mid-IR and/or far-IR wavelength such that the target macromolecule is directly disrupted and the target pathogen is neutralized by the exposure. Mid-IR and/or far-IR wavelengths can be selected to activate energy levels associated with the vibrational energy activation of certain types of bonds. According to one embodiment, the bond types range from covalent bonds, such as in a peptide chain, to weak hydrogen bonds such as in the partial intermolecular interaction H [symbol] and H [symbol] seen in folding phenomena. The excitation of one or more of the chemical bond structures in the biomolecules can lead to the following either new outcomes or relaxation to ground state.

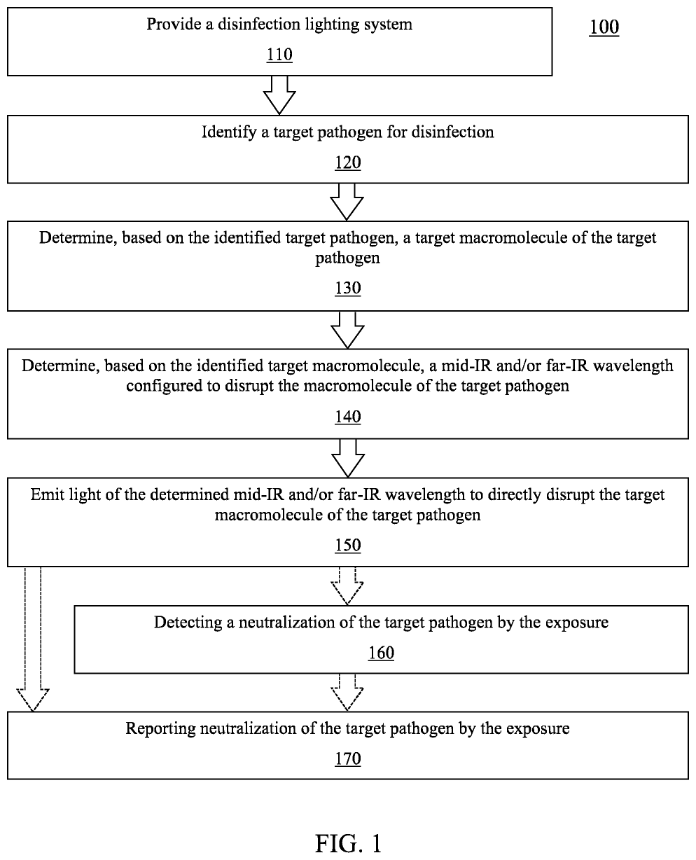

Referring to FIG. 1 is a flowchart of a method 100 for disinfection using a lighting system. At step 110 of the method, a lighting system is provided. The lighting system can be any of the systems described or otherwise envisioned herein. According to one embodiment, the lighting system comprises a light source or luminaire configured to emit a predetermined mid-IR and/or far-IR wavelength, and a controller configured to control the luminaire. The lighting system can optionally comprise many other elements or components. As described or otherwise envisioned herein, the lighting system can be a permanent installation or can be a handheld or other portable device, such as a personal device like a smartphone, tablet, or other device.

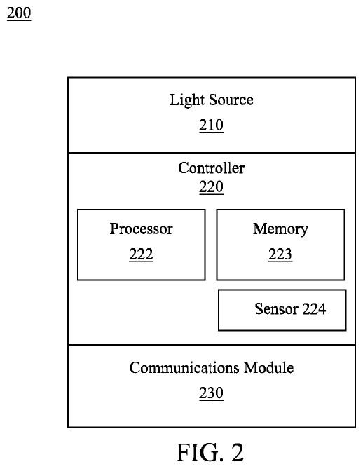

Referring to FIG. 2, in one embodiment, is a lighting system 200. The lighting system comprises one or more light sources 210. The one or more light sources 210 may be configured to emit light in the mid-IR and/or far-IR range, and may be configured to emit light in any other wavelength range. For example, there are IR solid state sources (such as LED or monolithic metal radiators) available on the market with up to 1 W emissive power. IR LEDs in the mid-range wavelength are commercially available, having center wavelengths from 1.9 μm (5263 cm⁻¹) to 7 microns (1428 cm⁻¹). Typical power levels are 10s to 1000s of microwatts. There are also emitters in the 2 μm (5000 cm⁻¹) to 16 μm (625 cm⁻¹) range. Similarly, there are IR emitters for spectroscopic analysis purposes such as those utilized in life science applications.

Lighting system 200 comprises a controller 220 configured to control one or more functionalities of the lighting system. The controller 220 may comprise a processor 222 programmed with software to perform one or more of the various functions discussed herein, and can be utilized in combination with a memory 223. Memory 223 can store data, including one or more commands or software programs for execution by processor 222, as well as various types of data including but not limited to one or more specific mid-IR and/or far-IR wavelengths configured to disrupt a target macromolecule of a target pathogen. For example, the memory 223 may be a non-transitory computer readable storage medium that includes a set of instructions that are executable by processor 222, and which cause the system to execute one or more of the steps of the methods described herein.

According to an embodiment, lighting system 200 may comprise a wired or wireless communications module 230 configured to communicate to another portion of the system, another system, or any other external source or structure. Accordingly, the communications module 230 may be directly wired to the other external source or structure, or the module may communicate via a wireless protocol such as Wi-Fi, Bluetooth, IR, radio, near field communication, and/or any other protocol.

Lighting system 200 also comprises a source of power, most typically AC power, although other power sources are possible including DC power sources, solar-based power sources, or mechanical-based power sources, among others. The power source may be in operable communication with a power source converter that converts power received from an external power source to a form that is usable by the lighting system. In order to provide power to the various components of the system, it can also include an AC/DC converter (e.g., rectifying circuit) that receives AC power from an external AC power source and converts it into direct current for purposes of powering the system's components. Additionally, the system can include an energy storage device, such as a rechargeable battery or capacitor, that is recharged via a connection to the AC/DC converter and can provide power to light source 210 and controller 220 when the circuit to AC power source is opened.

The lighting system 200 may comprise any other element or component. For example, the lighting system 200 may comprise a sensor 224 such as a motion detector configured to identify when the environment proximal the lighting system is empty or occupied. The lighting system 200 may comprise a temperature sensor 224 configured to measure a temperature of one or more of the one or more surfaces. The controller 220 can control the luminaire to: (1) stop emitting the mid-IR and/or far-IR wavelength if the measured temperature exceeds a predetermined threshold; or (2) lower an intensity of the mid-IR and/or far-IR wavelength if the measured temperature exceeds the predetermined threshold. This can be a direct or a remote temperature sensor 224 such as a thermopile. The sensor 224 of the lighting system 200 can detect neutralization of the target pathogen by the exposure. For example, the sensor 224 may be any sensor 224 configured for pathogen detection. In some embodiments, the lighting system 200 can include a timer configured to time exposure and detect neutralization of the target pathogen based on a time of exposure. Many other types of sensors, components, and elements are possible.

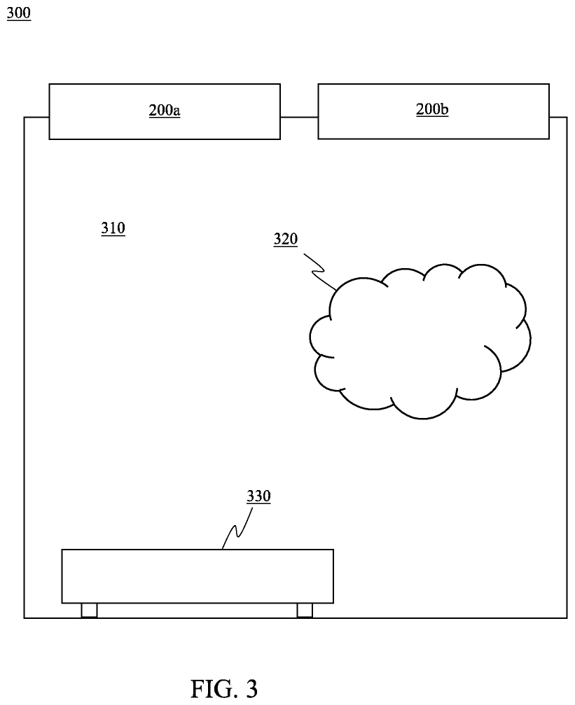

Referring to FIG. 3, in one embodiment, is an environment 300 which includes one or more lighting systems 200a, 200b. 200a and 200b may be two different lighting systems, or components of the same lighting system. In this example, the one or more lighting systems are ceiling structures. The ceiling structures can be any structure located near, within, or otherwise at the upper portion of the space 300. For example, the ceiling structures may be luminaires or other structures. There may be an optimal placement of a ceiling structure for disinfection of one or more volumes of air 320 and/or surfaces 330, for example, which may depend upon numerous factors such as the size and shape of environment 300. For example, the one or more lighting systems may be positioned to allow for the entire environment 300 to be disinfected, or may be positioned to allow for specific portions of the environment 300 to be disinfected. The installed configuration can be predetermined using maps, blueprints, or other information about the environment, or can be determined during installation and/or testing of the system. The configuration can later be modified or adjusted, for example, if the original placement is determined to be less than optimal, or if the use of the room changes over time. The space 310 may be any space for which surface and/or air disinfection is desired or required, such as an office, operating room, patient room, and/or any other room or space. For example, space 310 in FIG. 3 comprises a volume of air 320 and a piece of furniture or other object 330 for which pathogen disinfection may be desired.

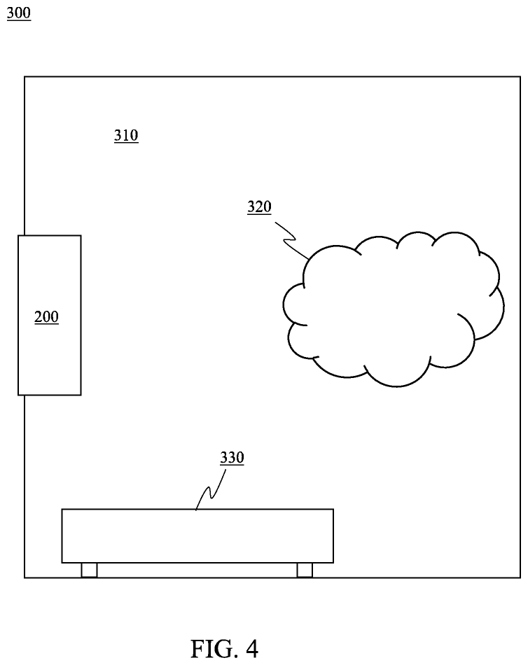

Referring to FIG. 4, in one embodiment, is an environment 300 which includes one or more lighting systems 200. In this example, the lighting system 200 comprises a wall-mounted structure with one or more light sources 210. The wall-mounted structure can be any structure located near, within, or otherwise associated with the wall or other portion of the environment. Although not shown, the lighting system 200 may be a floor-mounted or floor-embedded structure which has one or more light sources. The floor-mounted or floor-embedded structure can be any structure located near, within, or otherwise associated with the wall or other portion of the environment.

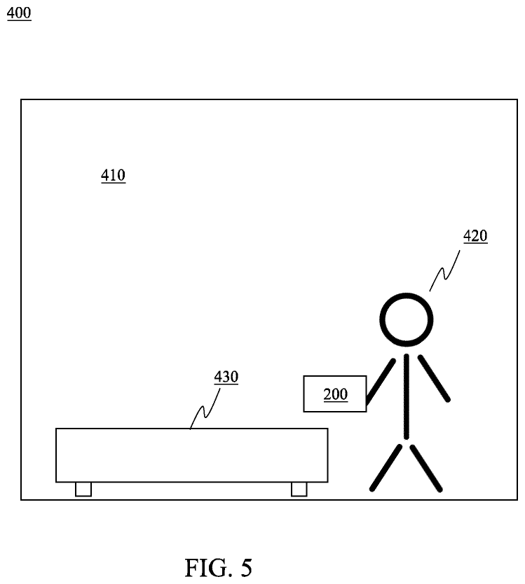

Referring to FIG. 5, in one embodiment, is an environment 400 which includes one or more lighting systems 200. In this example, the lighting system 200 comprises a handheld device utilized by a user 420 to disinfect one or more surfaces 430 in space 410. The surface may be any surface for which pathogen disinfection is desired or required. The lighting system 200 comprises a portable power source to allow user 420 to port the device from one location to another.

Returning to FIG. 1, at step 120 of the method a target pathogen is identified. The target pathogen is any pathogen for which a specific mid-IR and/or far-IR wavelength can target and disrupt a macromolecule of that pathogen, including but not limited to micro bacteria, viruses, prions, and other pathogens.

The identification of the target pathogen can be based on, for example, detection of the pathogen in a space or on a surface. For example, a space or surface may be periodically or continually monitored for the existence of one or more pathogens, either for a specific pathogen or any known pathogen. Alternatively, the system may be configured to target a specific pathogen regardless of whether the pathogen is detected in a space or on a surface. For example, the lighting system can be programmed or otherwise designed to periodically target one or more specific pathogens as described or otherwise envisioned herein. Thus, the system may be pre-programmed to target one or more specific pathogens, or the system may be modified, programmed, or otherwise designed after installation to target one or more specific pathogens.

At step 130 of the method, a target macromolecule of the identified target pathogen is identified. According to an embodiment, the target macromolecule is DNA, RNA, and/or a protein of the target pathogen. According to another embodiment, the target macromolecule is a lipid and/or a carbohydrate. The target macromolecule can be selected based on any criteria for selecting a target macromolecule. For example, the target macromolecule may be selected on the highest probability of successful disruption using a mid-IR and/or far-IR wavelength, ease of disruption, the lowest or minimal energy required for disruption, and/or any other criteria.

At step 140 of the method, a specific mid-IR and/or far-IR wavelength configured to disrupt the identified macromolecule of the target pathogen is determined. According to an embodiment, the specific mid-IR and/or far-IR wavelength is based on a spectroscopic property of the target pathogen.

For example, macromolecules such as DNA, RNA, and proteins absorb energy in the mid-IR and/or far-IR wavelength. DNA, for example, comprises one or more functional (vibration) groups for absorption. Absorption phenomena by the macromolecule can lead to, depending on the symmetry of the molecule, IR absorption leading to a dipole moment change during vibration, and/or Raman absorption leading to a change in the polarization of the molecule during the vibration.

According to an embodiment, for IR absorption, the excitation of bonds leads to oscillations of the bonds with a vibrational frequency, which is equal to the frequency of the absorbed radiation. In the case of Raman absorption, the two frequencies are different. Thus, specific mid-IR and/or far-IR wavelengths can be selected to excite specific bond types in specific macromolecules targeted for disinfection purposes. According to an embodiment, various disruption types target various bond types.

Since the target macromolecules are complex in nature, various chemical bonds and/or bonding interactions can be targeted. For example, proteins comprise 3D arrangements of amino-acid chains molecules, also with primary till quaternary structures, lipids are smaller molecules that form structures such as membranes for microbiological species, and carbohydrates can for example serve the role as structural component. Thus these form suitable target macromolecules.

Depending on the vulnerability of the target macromolecules towards vibrational energy activation of certain types of bonds, wavelengths can be selected as to activate energy levels associated to that bond. According to an embodiment, the bond types range from covalent bonds, such as in a peptide chain, to weak hydrogen bonds such as in the partial intermolecular interaction H 0 and H N seen in folding phenomena. The excitation of one or more of the chemical bond structures in the biomolecules can lead to the following either new outcomes or relaxation to ground state. According to an embodiment, a return to ground state is the aim when using IR radiation in a detection mode during analytical IR spectral analysis, where one analyses the composition of a sample in test and the percentages of a molecule in such sample. In such analysis method the excitation energies (intensities) are always chosen such as not to disturb the sample (prevent heating or chemical reactions).

However, for targeted disruption of macromolecules the goal is the opposite, namely that mid-IR and/or far-IR light is used to create radiation conditions causing thermal effects or induce chemical reactivity. This means that bond strength must be overcome by directly or indirectly providing energy transmission to the bond. According to an embodiment, one approach for disrupting a macromolecule comprises internal heating inside the molecule with IR light, in which non-radiative dissipation of the absorbed IR photo-energy leads to local thermal energy generation. According to an embodiment, this might be used as alternative for heat-based destruction of the molecule, now from the inside of the molecule, without affecting the surroundings/surfaces.

According to an embodiment, the IR light may result in reconfiguration of the tertiary/quaternary structure of a membrane and/or surrounding functional proteins. For example, this may be accomplished by targeting the weak intra- or intermolecular bonds. This may, for example, be utilized to modify the S-protein of a virus and thus prevent the recognition (ACE2 for COVID-19) on the host cell membrane (typically an endothelial cell), as just one specific example. Another approach would be to modify the interaction of the RNA chain with its protein envelope to disturb the protective shield of the virus, thus leading to pathogen inactivation.

According to an embodiment, the IR light may affect the secondary structure of RNA molecule and/or proteins. As an example, there may be an RNA chain interactions such as the folding between complementary regions, leading to inactivation of the RNA in later reproduction in the host cell.

According to an embodiment, the IR light may affect the primary structure of RNA molecule and/or proteins. As an example, the IR light may have an impact on a primary RNA or DNA chain, an impact on peptide bonds in proteins, and may lead to inactivation of the RNA in later reproduction, or destruction of properties or functions of proteins.

Thus, according to an embodiment, exposing a macromolecule to mid-IR and/or far-IR light can be utilized to permanently disrupt the macromolecule, such as disturbing the protein envelope of a virus and stopping its recognition function, or impacting RNA, DNA, or other macromolecules that form the effective pathogen.

According to another embodiment, exposing a macromolecule to mid-IR and/or far-IR light may result in a relaxation of the macromolecule to an original state after excitation. Accordingly, a primary excitation state can be followed by a second actuation with another wavelength that specifically targets the excited state absorption band, as to make sure that conformality of the protein or other macromolecule is permanently lost rather than returning to a ground state without modification. For example, recombination of the macromolecule can lead to another configuration which is not recognized by a host cell acceptor. Accordingly, step 140 may comprise determining a second mid-IR and/or far-IR wavelength configured to disrupt the target macromolecule in the intermediate product state, thus requiring a second exposure of the target macromolecule in the intermediate product state after an initial exposure. Alternatively, the second exposure may utilize a wavelength in the UV range, for instance 254 nm or 222 nm light at a moderate dose.

According to an embodiment, the mid-IR and/or far-IR wavelength is determined based at least in part on molecular modeling of the target macromolecule. Molecular modeling methods can be used to model or mimic the behavior of a potential or identified target macromolecule, thus allowing a determination of the best mid-IR and/or far-IR wavelength for disruption. There are many methods and approaches for the atomistic level description of a macromolecule, which enables a determination of one or more possible mid-IR and/or far-IR wavelengths for disruption. In embodiments, the system can use molecular modeling to determine the properties of the target molecule, including but not limited to bond strength, and intra- or intermolecular bond information. In some embodiments, the molecular modeling can determine a bond strength between at least two atoms (e.g., hydrogen atoms in different molecules). The system can use the determined bond strength and the molecular modeling to determine a mid-infrared (IR) and/or far-IR wavelength for breaking the bond of the target macromolecule of the target pathogen and to disrupt the target molecule. Once one or more possible mid-IR and/or far-IR wavelengths for disruption are identified, they can be experimentally tested and/or programmed into the lighting system controller for disinfection use. For example, the system can perform molecular modeling to determine a response to an applied mid-infrared (IR) and/or far-IR wavelength to determine, for example, if a bond was weakened, if a bond was broken, if an intra- or intermolecular bond was weakened or broken. The molecular modeling can mimic the response or behavior of the target macromolecule to the applied wavelength to determine if the dosage was enough or correct to break the bond and allow for disruption. The system can select an appropriate mid-IR and/or far-IR wavelength based on the molecular modeling.

According to an embodiment, in addition to determining a mid-IR and/or far-IR wavelength, step 140 may further comprise determining a dosage, which can be an amount of time required to expose the macromolecule of the target pathogen to the determined wavelength. This amount of time may be experimentally and/or theoretically derived, and may be determined based on how much energy for how long is required to create the molecular disruption. According to an embodiment, the time period for exposure may be a constant time period or an intermittent or pulsed time period. According to an embodiment, the time period for exposure may be nanoseconds, seconds, or longer.

According to an embodiment, the dosage is determined based at least in part on molecular modeling of the target macromolecule. Molecular modeling methods can be used to model or mimic the behavior of a potential or identified target macromolecule, thus allowing a determination of the dosage necessary for exposure to the determined mid-IR and/or far-IR wavelength to allow for disruption. There are many methods and approaches for the atomistic level description of a macromolecule, which enables a determination of the dosage. Once a possible dosage is determined, it can be experimentally tested and/or programmed into the lighting system controller for disinfection use. For example, the system can perform molecular modeling to apply one or more different dosages and determine a response to each of the different dosages of an applied mid-infrared (IR) and/or far-IR wavelength. The molecular modeling can mimic the response or behavior of the target macromolecule to each of the different dosages of the applied wavelength to determine if the respective dosage was enough or correct to break the bond and allow for disruption. The system can select an appropriate dosage of a mid-IR and/or far-IR wavelength based on the molecular modeling.

According to just one embodiment, a particular dosage may be necessary for a reason other than just an amount of time for exposure to deactivate or otherwise disrupt the macromolecule of the pathogen. For example, a pathogen may be covered or otherwise obscured or blocked by something such as dust, liquid, or another compound. This may necessitate a longer dosage to compensate for the microshadowing of the pathogen. As another example, where the pathogen is immersed in water or another fluid or liquid, dosage will be impacted: (1) because of possible attenuation of the radiation, and (2) because of possible relaxation (energy loss) due to interaction of the excited macromolecule with its environment. Many other factors may play into a determination of the dosage.

According to another embodiment, step 140 of the method comprises determining a specific near-IR wavelength configured to disrupt the identified macromolecule of the target pathogen is determined. According to an embodiment, the specific near-IR wavelength is based on a spectroscopic property of the target pathogen. According to an embodiment, the wavelength ranges for near-infrared, mid-infrared, and far-infrared can vary slightly among different informational sources. However, according to one embodiment, near-infrared can be the wavelength range of approximately 0.7 to approximately 3-5 μm, mid-infrared can be the wavelength range of approximately 3-5 μm to approximately 25-50 μm, and far-infrared can be the wavelength range of approximately 25-50 μm to approximately 200-350 μm. According to this embodiment, the target pathogen is exposed to the determined near-IR, optionally subject to a determined dosage, pursuant to step 150 and/or subsequent steps of the method.

At step 150 of the method, the lighting system 200 is activated or otherwise controlled to cause the light source to emit light of the determined mid-IR and/or far-IR wavelength to disrupt the target macromolecule of the target pathogen, such that the target macromolecule is directly disrupted and the target pathogen is neutralized by the exposure.

According to an embodiment, the light source is a component of a handheld device and surfaces and/or volumes of air can be exposed in closer proximity for disinfection. Thus, according to an embodiment, the light source is a component of a handheld and/or wearable device and is positioned at 10 cm or less from the target surfaces and/or volumes of air to be disinfected during emission of the determined mid-IR and/or far-IR wavelength. For example, the system may be utilized to disinfect surfaces such as electronic handheld devices.

At optional step 160 of the method, a sensor 224 of the lighting system detects neutralization of the target pathogen by the exposure. For example, the sensor 224 may be any sensor 224 configured for pathogen detection. In some embodiments, the lighting system can include a timer configured to time exposure and detect neutralization of the target pathogen by the exposure (e.g., time or length of exposure). Thus, at optional step 170 of the method, the system may be configured to report neutralization of the target pathogen by the exposure, or to report an attempt at neutralization of the target pathogen by the exposure. For example, the system or a component in communication with the system such as a user interface may be configured to report exposure of a surface or volume of air to the determined mid-IR and/or far-IR wavelength. The report may comprise one or more of the target macromolecules, the target pathogen, the one or more determined mid-IR and/or far-IR wavelength, the one or more periods of time, and an outcome of the exposure, among other information. As another example, the user interface may convey a message such as ‘end of disinfection—safe’, ‘not sufficiently disinfected—unsafe’, and so on, and this can be done by using a LED indicator among other indicators.

According to an embodiment, the disinfecting lighting system is mobile within the disinfection environment. For example, the disinfecting lighting system may be or may comprise a mobile element such as a robot or a drone, or a fixed but moveable system that can bring the light source within the necessary proximity to target surfaces or other target items for disinfection.

According to an embodiment, the disinfecting lighting system can be combined with other disinfection means for complementary or reinforced disinfection. These other disinfection systems may be, for example, safe for individuals.

According to an embodiment, the system and/or method may further comprise eliminating at least some liquid surrounding the target pathogen prior to the step of exposing the pathogen or surface to the mid-IR and/or far-IR wavelength. This may, for example, improve the absorption of the energy by the target macromolecule, as compared to heating water molecules surrounding the pathogen. Eliminating at least some liquid surrounding the target pathogen may comprise an active and/or a passive elimination. For example, the surface may be actively dried, or the system may wait a predetermined or experimentally-derived or tested amount of time for liquid on surface and/or surrounding the pathogen to be sufficiently eliminated. Further, according to an embodiment the liquid may be an aerosolized liquid that occludes the virus.

According to an embodiment, the system and/or method may further comprise an algorithm, lookup table, or other component configured to define a mid-IR and/or far-IR wavelength that will or should be used to target a class of pathogens or a specific pathogen. For example, a user may determine that a surface is contaminated with or likely contaminated with one or more specific pathogens or types of pathogens. If the system comprises a lookup component comprising the wavelengths recommended to target those one or more pathogens, the user can either select that wavelength as an option or otherwise program lighting system 200 to emit the recommended wavelength. This may be accomplished, for example, via the user interface either by selecting a recommended wavelength already found within the system, or manually entering a recommended wavelength, among other options.

According to an embodiment, the lighting system 200 comprises a temperature sensor 224 configured to measure a temperature of one or more of the one or more surfaces. This can be a direct or a remote temperature sensor 224 such as a thermopile. The measured temperature can be utilized to ensure that the surface and/or volume or air remains within a specified temperature range. According to another embodiment, the system can be configured or designed to detect a temperature fluctuation or change due to the presence of a non-pathogen living thing such as a human or pet within the environment. Accordingly, the controller can be configured to control the luminaire to: (1) stop emitting the mid-IR and/or far-IR wavelength if the measured temperature exceeds a predetermined threshold or if the system detects the presence of a person or animal; or (2) lower an intensity of the mid-IR and/or far-IR wavelength if the measured temperature exceeds the predetermined threshold or if the system detects the presence of a person or animal. According to another embodiment, the system can be configured or designed to operate in a pulsed mode or otherwise designed to prevent or limit as much harmful exposure to humans or other non-pathogen living things as possible.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

1. A method for using a lighting system, comprising:
determining a wavelength configured to disrupt a target macromolecule of a target pathogen, wherein the target macromolecule is DNA, RNA, and/or a protein of the target pathogen;
exposing, via a light source of the lighting system, the target pathogen to the determined wavelength, wherein the target macromolecule is directly disrupted and the target pathogen is neutralized by the exposure; and
detecting, by a sensor of the lighting system, neutralization of the target pathogen by the exposure.

2. The method of claim 1, wherein the wavelength includes a mid-infrared (IR) and/or far-IR wavelength configured to disrupt a target macromolecule of a target pathogen is determined based on a spectroscopic property of the target pathogen.

3. The method of claim 1, further comprising the step of determining a dosage at which to expose the target pathogen to the determined wavelength.

4. The method of claim 1, wherein the mid-IR and/or far-IR wavelength and/or a dosage is determined based at least in part on molecular modeling of the target macromolecule.

5. The method of claim 1, further comprising the step of eliminating at least some liquid surrounding the target pathogen prior to said exposing step.

6. The method of claim 1, wherein the macromolecule is a surface protein of a virus.

7. The method of claim 1, wherein exposing the target pathogen to the determined wavelength results in an intermediate product state of the target macromolecule leading to deactivation of the target macromolecule, and further comprising the steps of:
determining a second wavelength configured to disrupt the target macromolecule in the intermediate product state; and
exposing the target pathogen to the determined second wavelength.

8. The method of claim 1, further comprising the step of reporting neutralization of the target pathogen by the exposure.

9. The method of claim 1, wherein exposing the target pathogen to the determined wavelength comprises exposing a volume of air to the light source of the lighting system.

10. The method of claim 1, wherein exposing the target pathogen to the determined wavelength comprises exposing a surface which is a first distance from the light source of the lighting system, wherein the first distance comprises at least a meter.

11. A lighting system configured to neutralize a target pathogen, comprising:
a light source configured to emit a predetermined wavelength, wherein the wavelength is configured to disrupt a target macromolecule of the target pathogen, wherein the target macromolecule is DNA, RNA, and/or a protein of the target pathogen;
a controller configured to control the light source, wherein the controller is pre-programmed with the predetermined wavelength; and
a sensor configured to detect neutralization of the target pathogen.

12. The lighting system of claim 11, wherein the predetermined wavelength results in an intermediate product state of the target macromolecule, and wherein the light source is configured to emit a second predetermined wavelength, the second wavelength configured to disrupt the target macromolecule in the intermediate product state, and wherein the controller is further pre-programmed with the second predetermined wavelength.

13. The lighting system of claim 11, wherein the lighting system is configured to neutralize a target pathogen located on one or more surfaces of an environment in which the lighting system is installed.

14. The lighting system of claim 13, further comprising a temperature sensor configured to measure a temperature of one or more of the one or more surfaces, and wherein the controller is further configured to control the light source to: (1) stop emitting the wavelength if the measured temperature exceeds a predetermined threshold; or (2) lower an intensity of the wavelength if the measured temperature exceeds the predetermined threshold.

15. A handheld device configured to neutralize a target pathogen, comprising:
a light source configured to emit a predetermined wavelength, wherein the wavelength is configured to disrupt a target macromolecule of the target pathogen, wherein the target macromolecule is DNA, RNA, and/or a protein of the target pathogen; and
a controller configured to control the light source, wherein the controller is pre-programmed with the predetermined wavelength, wherein the predetermined wavelength is determined based at least in part on molecular modeling of the target macromolecule.

16. The handheld device of claim 15, wherein the handheld device is configured to expose the target pathogen to the determined wavelength at a distance of 10 cm or less.
